SEQUENCE LISTING (10 sequences)

SEQ ID NO: 1 - DNA, 17 bp, Artificial Sequence
  Primer
cccgtggccttcaaggt

SEQ ID NO: 2 - DNA, 20 bp, Artificial Sequence
  Primer
cgttacccgccatgacagta

SEQ ID NO: 3 - DNA, 24 bp, Artificial Sequence
  Primer
ccagaagaactggtacatcagcaa

SEQ ID NO: 4 - DNA, 21 bp, Artificial Sequence
  Primer
cgccatactcgaactggaatc

SEQ ID NO: 5 - DNA, 20 bp, Artificial Sequence
  Primer
agcaaaggtgcagcctttgt

SEQ ID NO: 6 - DNA, 19 bp, Artificial Sequence
  Primer
gcgcctgggtctcttcact

SEQ ID NO: 7 - DNA, 21 bp, Artificial Sequence
  Primer
gccgaccaaggaaaactcact

SEQ ID NO: 8 - DNA, 20 bp, Artificial Sequence
  Primer
ggcacaggtgatgcctagga

SEQ ID NO: 9 - DNA, 19 bp, Artificial Sequence
  Primer
aatctccaggggcaccatt

SEQ ID NO: 10 - DNA, 18 bp, Artificial Sequence
  Primer
cgttggctcccactttgt

(19) United States
(12) Patent Application Publication
(10) Pub. No.: US 2011/0117521 A1
(43) Pub. Date: May 19, 2011

(54) BIOLOGICAL REGENERATE

(75) Inventors: Jyrki Törnvall, Espoo (FI); Bettina Lindroos, Tampere (FI); Marjo-Riitta Suuronen, Pirkkala (FI); Susanna Miettinen, Siivikkala (FI); Karri Mesimäki, Helsinki (FI); Jari Mauno, Helsinki (FI)

(73) Assignee: Tampereen yliopisto, Tampere (FI)

(21) Appl. No.: 12/622,108

(22) Filed: Nov. 19, 2009

(51) Int. Cl. A61F 2/28; C12N 5/071; C12N 5/077; A61C 8/00

(52) U.S. Cl. 433/173; 435/395; 623/17.17

(57) ABSTRACT

The present invention relates to the field of microvascular reconstruction surgery of bone defects. More specifically, the invention relates to a biological jaw regenerate and to methods of preparing said jaw regenerate. Furthermore, the invention relates to a method of reconstructing a jaw defect in a patient in need thereof.

### FIELD OF THE INVENTION

The present invention relates to the field of microvascular reconstruction surgery of bone defects. More specifically, the invention relates to a biological jaw regenerate and to methods of preparing said jaw regenerate. Furthermore, the invention relates to a method of reconstructing a jaw defect in a patient in need thereof.

### BACKGROUND OF THE INVENTION

The most commonly used surgical technique for reconstructing a major bony defect is harvesting of autologous bone causing severe donor site morbidity and risk of infection.

New reconstructive techniques are developing, and bone has been employed as a supportive scaffold. Moghadam et al. reported in J. Craniofac. Surg. 12: 119-27, 2001 a case of mandibular defect reconstruction using native bone morphogenetic protein (BMP) bioimplant, poloxamer-based gel and allogenic bone blocks. Warnke et al. published in Lancet 364:766-70, 2004 an article were a mandibular defect was repaired using bovine collagen and bovine bone mineral blocks, recombinant human (rh) BMP-7, and autologous bone marrow. Furthermore, Lendeckel et al. reported in J. Craniomaxillofac. Surg 32:370-3, 2004 a case reconstructing a calvarial defect utilizing adipose tissue derived mononuclear cells with patient's own cancellous bone and autologous fibrin glue.

Reconstructive surgery is evolving into a multidisciplinary field were reconstructive surgeons work in collaboration with scientists and engineers. Reconstructive surgery requires alternate approaches that may, at least, decrease the level of side effects associated with surgery. While the elimination of surgery is unrealistic, new technologies may decrease the number of procedures and may also improve the outcome of the necessary surgeries.

International patent publication WO 2006/089359 discloses a bone replacement tissue consisting of a cone- or cup-shaped biocompatible scaffold containing osteoblast progenitor cells, and grown in vivo in a host tissue in order to induce osteogenesis and angiogenesis prior to translocation into a desired position in a patient.

Craniofacial reconstruction sets special functional and aesthetic demands for bone regenerates, especially when involving the dental reconstruction. Thus, new methods and means for jaw reconstruction meeting the clinical cell therapy safety standards of the European Union are still needed.

### BRIEF DESCRIPTION OF THE INVENTION

The present invention is based on clinical studies for microvascular reconstruction surgery of maxillary and mandibular bone defects. By the means and methods provided herein it is possible to significantly shorten the healing and toothless period after oral surgery.

The present invention relates to a biological jaw regenerate comprising a biocompatible scaffold inoculated with osteogenic cells. In some embodiments, the regenerate may further comprise at least one dental implant for attaching dental prosthesis.

In some embodiments, the scaffold comprises a frame filled with a bone substitute material. In some further embodiments, the frame is an inert metal cage, such as a titanium cage, and the bone substitute material is beta-TCP preferably in the form of granules. In other embodiments, the scaffold is a homogenous structure made of a biocompatible material.

Suitable osteogenic cells for use in the regenerate of the present invention include mesenchymal stem cells, such as adipose stem cells, i.e. mesenchymal stem cells obtained from autogenic or allogenic fat.

Optionally, the regenerate according to the present invention may comprise osteoinductive growth factors, such as bone morphogenetic proteins (BMPs) including but not limited to BMP-2 and/or BMP-7.

Furthermore, the present invention relates to a method of preparing a jaw regenerate according to the present invention and all its embodiments. The method comprises a) providing a biocompatible scaffold shaped to fit into the jaw region where the regenerate is required, and b) inoculating said scaffold with osteogenic cells to form a biological jaw regenerate. The method may further comprise adding osteoinductive growth factors such as bone morphogenetic proteins (BMPs) including but not limited to BMP-2 and/or BMP-7. Optionally, the scaffold of the regenerate may comprise fastened thereto at least one dental implant for a dental prosthesis.

The present invention also relates to a method of reconstructing a jaw defect, such as a maxillary or mandibular defect, in a patient in need thereof. The method comprises a) providing a biological jaw regenerate according to the present invention and any embodiment thereof, b) inserting the biological jaw regenerate into a pouch in an autogenic muscle, c) allowing formation of microvasculature in the biological jaw regenerate, d) relocating the regenerate and its microvasculature to the jaw region where the regenerate is required. Optionally, the scaffold may comprise fastened thereto at least one dental implant for a dental prosthesis, and the method may thus further comprise a step of attaching a dental prosthesis to the dental implant between the steps c) and d).

### BRIEF DESCRIPTION OF THE DRAWINGS

FIG. 1 is a schematic outline of the bone reconstruction procedure.

FIG. 2 illustrates a postoperative status of patient 1 after two months. FIG. 2A shows that rectus abdominis muscle has atrophied nearly totally and epithelialized almost completely. Only a small area in a molar region was non-epithelialized. Note the well formed buccal sulcus. Axial (FIG. 2B) and 3D CT-scans (FIG. 2C) show the shape and normal bone density of the new maxilla.

FIG. 3 shows dental implants of patient 1 placed 4 months after the bone and soft tissue reconstruction.

FIG. 4A illustrates a preoperative clinical status of patient 1 after 28 months of removal of keratocyst by hemimaxillectomy. FIG. 4B illustrates the final clinical status of patient 1 one year after the bone and soft tissue reconstruction, with temporary dental implant rehabilitation.

### DETAILED DESCRIPTION OF THE INVENTION

The invention is based on clinical studies where ectopic bone formation inside a muscle free flap has been performed to reconstruct a bony defect.

The invention relates to custom-made biological regenerates, especially to maxillary and mandibular jaw regenerates. Herein, a biological regenerate refers to a living implant or transplant that may be used for replacing autogenic or allogenic body parts.

The biological jaw regenerate according to the present invention comprises a 3D scaffold inoculated with osteogenic cells.

Herein, osteogenic cells refer to osteoblast and cells having an ability to differentiate into osteoblasts and undergo osteogenesis. Such cells include, but are not limited to, mesenchymal stem cells (MSCs), which are multipotent adult stem cells capable of differentiating into a variety of cell types. Methods of isolating MSCs from different sources including bone marrow and adipose tissue have been established and are readily available. Autogenic MSCs are isolated from the patient's own tissue, whereas allogenic cells are isolated from an organism other than the patient. Both autogenic and allogenic MSCs may be used in different embodiments of the biological jaw regenerate.

While the bone marrow MSCs are a viable option for a stem cell population in bone regeneration, there are disadvantages that must be recognized. In order to use stem cells as clinical treatment, there has to be means to isolate cells in large enough quantities. In a bone marrow aspiration the cell yield is usually low compared to the cell yield from adipose tissue.

Autogenic adipose stem cells (ASCs) are an attractive alternative for bony reconstruction. They can be obtained in large quantities, are immunologically inactive and do not transfer infectious agents. Furthermore, when the autologous cells are handled and prepared without animal derived material in Good Manufacturing Practice (GMP) standard clean rooms, the cells can be considered safe for clinical cell therapy applications.

Embryonic stem cells are pluripotent cells being able to differentiate into a wide variety of different cell types. Thus, they are an alternative as osteogenic cells in the present regenerates. Methods of obtaining embryonic stem cells are available in the art. Blastomere biopsy is an attractive new technology which allows isolation and propagation of embryonic stem cells without damaging the donor embryo.

Induced pluripotent stem cells (iPSCs) are another alternative for use in the present regenerates. iPSCs are generated from differentiated cells, typically from adult somatic cells such as fibroblasts by developmental reprogramming. Such cells have been described e.g. in WO 2008/151058 and US 2008/076176.

Other sources of osteogenic cells may become available as the technology advances. Osteogenic differentiation may be determined easily e.g. by biochemical alkaline phosphatase staining, as is well known in the art.

The cell isolation and expansion is carried out according to GMP standards without animal-derived reagents. As part of the GMP quality control, the osteogenic cells are analyzed for sterility and endotoxins and are also tested for mycoplasma contamination. Additionally, the cells may undergo an extensive panel of in vitro analyses, including surface marker characterization, differentiation analyses, and adhesion and viability studies.

The scaffold used in the present regenerate is shaped so as to fit into the region where the regenerate is required. Anatomical modeling may be required prior to shaping the scaffold manually or by a rapid prototyping. Methods and means for the modeling and shaping are available in the art.

The scaffold material must be biocompatible. Herein, a biocompatible material refers to a safe and non-toxic material which does not elicit any undesirable local or systemic effects in the host. Examples of biocompatible materials include inert metals such as titanium and titanium alloys, bioactive glasses and glass ceramics.

The scaffold may be provided as a biocompatible frame filled with a bone substitute material. Preferably, the bone substitute material is in the form of granules so as to allow even distribution of inoculated osteogenic cells.

The frame provides mechanical support to the biological jaw regenerate as well as determines the shape thereof. The frame may be provided as an inert metal cage, such as a titanium or titanium alloy cage. One disadvantage associated with the metal cage is that it may be exposed in the course of time. Such a risk may be avoided by replacing the metal cage with a biodegradable frame or cage structure, especially in the alveolar crest region of the jaw regenerate. Suitable biodegradable frame and cage materials include, but are not limited to, polylactide acid and composites of different biodegradable materials. Herein, biodegradable materials refer to materials which degrade in the body and are replaced with newly regenerated tissue. In this case, the biodegradable materials are replaced with regenerated bone.

Osteoinductive materials may be used as a bone substitute material inside the frame. Herein, an osteoinductive material refers to any material that is able to induce osteogenesis, i.e. bone formation, of osteogenic cells. Examples of osteoinductive materials include bioactive glass and glass ceramics.

Beta-tricalcium phosphate (beta-TCP) is a well known osteoinductive material suitable for use as bone substitute material. It has been extensively studied and clinically used because its molecular composition is similar to that of human bone. Among bioceramics, beta-TCP has an excellent osteoconductivity, bioactivity and ability to form a strong bone-calcium phosphate interface. In general, clinical application of beta-TCP is limited due to its poor mechanical properties, in particular its low fracture toughness, and its improper degradation properties. These properties do not, however, hinder its use inside a supportive frame since no mechanical properties or fast degradation are required.

In one preferred embodiment, the regenerate consists of a titanium cage filled with a mixture of ASCs and beta-TCP granules.

An osteoinductive material suitable for use in the present invention may also contain a combination of various biomaterials. Examples of such combinations include mixtures of a bioactive glass or tricalcium phosphate with collagen, poly(lactic-co-glycolic acid) (PLGA) and/or polylactic acid (PLA). Other suitable combinations are known to a person skilled in the art.

In some embodiments, the scaffold may be a homogeneous solid or semisolid structure made of any suitable biodegradable material. In such cases, the scaffold may be cut to its final shape and no separate frame structure is required. The osteogenic cells may be provided on the surface of the structure or as integrated into the structure. In further embodiments, the structure may be porous.

Optionally, the biological regenerate may be supplemented with growth factors such as bone morphogenetic proteins (BMPs). BMP-2 and/or BMP-7 are preferable due to their osteoinductive activity. Recombinant human (rh) growth factors may be used in order to avoid animal derived materials. Growth factors may be administered in the cell culture phase and/or they may be added to the regenerate prior to insertion into a muscle and/or at the time of final implantation into the jaw region. A person skilled in the art can easily determine suitable growth factor concentrations. In some embodiments, ASCs are cultured in the presence of about 1 mg to about 100 mg, preferably about 5 mg to about 20 mg, more preferably about 10 mg to about 15 mg, and even more preferably 12 mg rhBMP-2, for a period of time from a few minutes to several weeks, preferably from about 24 hours to about 72 hours, and more preferably for about 48 hours, prior to mixing with a bone substitute material.

In cases were osteogenic differentiation of inoculated cells is induced by growth factors such as BMP-2, the bone substitute material inside the frame needs not be osteoinductive, but acts just as a carrier of osteogenic cells into a defect site.

So far, seven patients suffering from a craniofacial bone defect have been successfully treated according to the present invention. These patients have now been followed up for 6 to 24 months without any unexpected complications. Unfortunately, one further patient (patient 2 in the examples) has lost his regenerate due to insufficient ossification. Special care will be taken in the future in monitoring ossification of the regenerate.

It has now unexpectedly been found that the biological jaw regenerate may also comprise dental implants, i.e. artificial teeth roots. It is surprising that the regenerate is strong enough to allow instant dental implant rehabilitation. However, it may be necessary to provide the regenerate first with temporary e.g. acrylic prosthesis to provide gradual loading and stimulation of the new bone regenerate and to avoid too hard loading. With the use of the temporary prosthesis or a specific surgical guide the positioning of the bone regenerate is easier and more precise which also shortens the operation time.

Later the temporary prosthesis is replaced with a final prosthesis which is designed according to the maturated soft tissue and preliminary plan. This way the duration of healing period after oral/maxillofacial surgery is shortened to the same time span which is necessary for the general osteosynthesis period and the patient has the temporary prosthesis in use during this healing period, since the maturation of bone regenerate and osseointegration of dental implants occur at the site where the reconstruct is placed for maturation.

The planning of the regenerate is based on both clinical and radiological examination and evaluation of the initial occlusal situation. Three dimensional radiological examination is necessary for exact evaluation of bone structure and the size and shape of bone defect and precise determination of osteotomy lines.

During treatment planning the exact positions of final tooth crowns are determined concerning both functional and aesthetic aspects. When the occlusion plane or the position of dental arch need to be altered at the positioning of the tissue engineered jaw regenerate, the final predicted position of teeth are scanned in centric relation and this three dimensional view is used in determining the final shape and volume of bone reconstruction.

The positioning of dental implants in the engineered jaw regenerate can be planned either manually with stereolithography model or by computer aided design (CAD) programs.

Prosthetic planning specifies the intended structure in detail: the final dental prosthesis can be produced as a screw retained or cemented fixed bridge or bar-supported removable hybrid denture. The necessary implants and abutments are chosen according to the planned construction and the angulation of implants and abutments is defined with the final tooth setup either on stone models or by CAD program. When the planning is done manually, implant replicas are fixed on the tooth setup to be transferred to the stereolithography model for confirmation of the plan and modeling of the framework for tissue-engineered bone. The area around implants is covered with sufficient amount of solid material, e.g. acrylic resin, to make a template for forming of the supporting frame and finally an inert supporting bar is manufactured from e.g. titanium, zirconia or gold alloy for splinting the implants into the desired positions. The total volume, size and shape of the frame are evaluated also regarding the effect to the facial soft tissues and if necessary implant positions or angulation are altered accordingly. The supporting frame is designed so that it has openings for the dental implants so that the dental implants can be inserted into the frame and the bar is fixed to the frame so that the frame and dental implants cannot move in relation to each other. The frame is made with provisions for later fixation to the proper place for the aimed repair of bone defect.

The planning of dental prosthesis, teeth location and implant sites can also be done by CAD and the manufacturing of the cage and fixation bar can be made by CAD/CAM method based on similar design. When the frame is made out of bioresorbable material the exact places for osteosynthesis devices, e.g. plates and screws, are marked with identifiable pins or marks for the exact positioning of the structure into its proper place.

The regenerate is provided with blood supply by microvascular reconstruction surgery. To this end, the regenerate is inserted carefully through an incision into a pouch prepared in the patient's muscle, e.g. rectus abdominis or latissimus dorsa, without disturbing the vascular supply of the muscle. Position of the construct inside the muscle is dependent on the shape and location of the bone defect. Furthermore, the position, shape and size of lacking soft tissue have to be taken into account. The implant has to be inserted inside the muscle so that the major blood vessels of the muscle and ossifying bone can be connected to the major blood vessels of the defect area without hindering the attachment of tooth implants and increasing the susceptibility for thrombosis. This means that the blood vessels have to be positioned on either side or underneath of the forming bone structure. The incision is closed in layers.

Ossification of the regenerate inside the muscle may be followed up e.g. by radiography (X-ray), computed tomography (CT), scintillography or histological analysis of a biopsy to make sure that the regenerate has ossified sufficiently prior to harvesting from the muscle. Depending on the size and shape of the regenerate, typically, six to nine months, is required for sufficient ossification.

After harvesting the regenerate from the muscle, teeth, preferably temporary acrylic teeth, are attached to the regenerate by dental implants. The regenerate containing dental implants and prosthesis is then fixed to its final place e.g. by titanium screws. Vascular anastomosis are done to the facial artery and vein end-to-end.

A temporary fixed bridge or surgical guide is made according the prosthetic plan to be used during the surgery when the tissue-engineered bone block is positioned into its place. The supporting bar used for fixing the dental implants to the frame of the regenerate is first removed and the bridge or surgical guide is fixed into its place. The occlusion is designed into exact intercuspal position and provisions, e.g. pins or hooks, are added to the frame so that intermaxillary fixation is possible during operation. In case of hybrid denture as the final prosthetic construction, the bar and surgical guide are made with suitable attachments.

Advantages of the present invention include diminishing patient morbidity and social disadvantages associated with a toothless healing period. Maturation of the jaw regenerate and osseointegration of the dental implants occur simultaneously in an autogenic muscle free flap thus shortening the oral healing period after reconstruction surgery. With conventional techniques ossification of a bone craft occurs only after reconstruction surgery and an additional operation is required for attaching dental implants thus prolonging the healing time and toothless period. Furthermore, positioning of conventional bone crafts in the jaw region as well as the final outcome is less accurate as compared to those of the present technology.

In some embodiments of the present invention, the regenerate may be placed directly into the defect area. In such cases one operation less is required because the regenerate matures and ossifies in its final position. In other words, no separate maturation period inside a muscle free flap involving inserting and harvesting of the regenerate is required. Excellent healing results have been obtained according to this embodiment.

It will be obvious to a person skilled in the art that, as the technology advances, the inventive concept can be implemented in various ways. The invention and its embodiments are not limited to the examples described below but may vary within the scope of the claims.

### EXAMPLES

The board of directors of the local hospital district gave their consent to these experimental studies. Prior to the surgeries, the patients were thoroughly informed about the procedures, which they approved and gave their written consents.

### Example 1

### Stem Cell Isolation, Preparation for Transplantation, and Analyses

Approximately 100 to 200 ml of subcutaneous abdominal fat was harvested in an operation and transported to REGEA Institute for Regenerative Medicine for stem cell isolation and expansion. Additionally, 50 to 60 ml of autologous serum was also obtained for the expansion of the autologous stem cells.

ASCs were isolated and expanded in vitro in GMP-class clean rooms according to Standard Operation Procedures at REGEA Institute for Regenerative Medicine. Isolation was carried out using mechanical and enzymatic isolation as described previously (Gimble et al., Cytotherapy 2003; 5:362-9; 28. Niemelä et al., J Craniofac Surg 2007; 18:325-35). Briefly, the adipose tissue was minced into small fragments and digested with collagenase type I (Invitrogen, Paisley, Scotland, UK) to separate the adipose derived stem cells from the surrounding tissue. The isolated cells were expanded 14 days in basal media (BM) containing DMEM/F-12 (Invitrogen, Paisley, Scotland, UK) with 15% of autologous serum, without antibiotics. Subsequently, the cells were detached with TrypLE Select (Invitrogen; patient 1) or mechanically (patient 2) and prepared for cell transplantation. Prior to combining the cells with beta-TCP (Chronos®, Synthes, Oberdorf, Switzerland, porosity 60%, granule size 1.4-2.8 mm), beta-TCP was incubated for 48 hours in BM containing 12 mg rhBMP-2 (InductOS, Wyeth Europa, Berkshire, UK). Following the incubation, the BM containing rhBMP-2 was discarded. Subsequently, to allow cell attachment, approximately 13×106 cells were combined with 60 ml of beta-TCP granules 48 hours prior to the operation. Cell sterility and endotoxins were tested at the Department of Public Health (University of Helsinki, Helsinki, Finland) according to methods described in European Pharmacopoeia (Council of Europe, Strasbourg). Furthermore, the cells were tested negative for mycoplasma contamination as determined by a mycoplasma PCR kit (VenorGem, Minerva Biolabs GmbH, Berlin, Germany).

Cell attachment to the beta-TCP granules and the cell viability were studied using Live/Dead staining prior to and after the surgical cell transplantation operation. Subsequently, the cell-biomaterial combination was incubated with a mixture of CellTracker™ green (5-chloromethylfluorescein diacetate (CMFDA), Molecular Probes, Eugene, Oreg., USA) and Ethidium Homodimer (EH-1) Molecular Probes). The viable cells (green fluorescence) and dead cells (red fluorescence) were detected with a fluorescence microscope. The cells proliferated rapidly in the patient's own serum in both patient 1 and 2. Viable and adherent ASC were transplanted with the beta-TCP granules into the patient were confirmed by Live/Dead staining for both patients.

For patient 1, all subsequent in vitro analyses were performed using BM supplemented with 15% human serum of the clot type AB (PAA Laboratories GmbH, Pasching, Austria), and antibiotic/antimycotic (a/a; 100 U/ml penicillin, 0.1 mg/ml streptomycin, and 0.25 μg/ml amphotericin B; Invitrogen). Since the amount of autologous serum was limited and rapid cell expansion for clinical use requires large amount of serum, commercial human serum was used for the following analyses. In the case of patient 2, the in vitro analyses were performed in autologous serum.

For both patient 1 and 2, ASCs were further expanded in vitro and were analyzed by flow cytometry (FACSAria®, BDBiosciences, Erembodegem, Belgium). Monoclonal antibodies against CD9-PE, CD10-PECy7, CD13-PE, CD29-APC, CD49d-PE, CD90-APC, CD106-PE-Cy5, CD146-PE and CD166-PE (BD Biosciences); CD45-FITC (Miltenyi Biotech, Bergisch Gladbach, Germany); CD31-FITC, CD34-APC and CD44-FITC (Immunotools GmbH Friesoythe, Germany); and CD105-PE (R&D Systems Inc, MN, USA) were used. Also, markers against major histocompatibility class I antigens (HLA-ABC), were analyzed. Analysis was performed on 10,000 cells per sample and the positive expression was defined as the level of fluorescence greater than 99% of the corresponding unstained cell sample. The surface marker analysis demonstrated that ASC population was homogenous morphology and showed a surface marker profile characteristic of ASCs, with positive expression for adhesion molecules CD9, CD29, CD49d, CD105, CD106, and CD166; receptor molecule CD44; surface enzymes CD10 and CD13, and extracellular matrix protein CD90, and major histocompatibility class I antigens (HLA-ABC). All cell sources lacked expression of CD31, CD34, CD45, CD146 and CD106 suggesting lack of cells of hematopoietic and angiogenic lineages (Table 1).

TABLE 1
Surface marker expression of undifferentiated ASCs analyzed by
flow cytometric analysis of 10,000 cells. Results are displayed as means
of the surface marker expression in percent

| | | Surface | Surface |
|---|---|---|---|
| | | marker | marker |
| | | Expression | Expression |
| Antigen | Surface Protein | Patient 1 | Patient 2 |
| CD 9 | Tetraspanin receptor | 7.6 | 10.4 |
| CD 10 | Common leukocyte lymphocytic | 99.9 | 98.5 |
| | leukemia antigen (CALLA) | | |
| CD 13 | Aminopeptidase N | 99.7 | 99.9 |
| CD 29 | Integrin β1 | 98.8 | 99.1 |
| CD 31 | Platelet endothelial cell adhesion | 0.4 | 0.4 |
| | molecule (PECAM) | | |
| CD 34 | Sialomucin-like adhesion molecule | 0.9 | 0.8 |
| CD 44 | Hyaluronate, | 86.5 | 93.0 |
| | Lymphocyte homing-associated | | |
| | cell adhesion molecule (HCAM) | | |
| CD 45 | LCA | 0.5 | 1.2 |
| CD 49d | Integrin α2, VLA-4 | 26.6 | 53.2 |
| CD 90 | T cell surface glycoprotein 1 (Thy-1) | 99.5 | 94.8 |
| CD 105 | SH-2, endoglin | 99.3 | 99.8 |
| CD 106 | Vascular cell adhesion | 0.4 | 0.7 |
| | molecule 1 (VCAM-1) | | |
| CD 146 | Melanoma cell adhesion molecule | | 1.6 |
| | (MCAM) | | |
| CD 166 | Activated leukocyte cell | 91.8 | 93.7 |
| | adhesion molecule (ALCAM) | | |
| HLA-ABC | Major histocompatibility class I antigens | | 68.6 |

Differentiation studies were performed only on patient 1. In the differentiation studies, all cultures were maintained for 14 days and all control cell cultures were maintained in BM. For the in vitro osteogenic differentiation analyses, surplus cells and biomaterial were maintained on a 6-well plate in osteogenic media (OM), containing BM supplemented 50 μM L-ascorbic acid 2-phosphate (asc.ac; Sigma), 10 mM beta-glycerophosphate (Sigma) and 100 nM dexamethasone (Sigma). The cultures were subsequently analyzed by alkaline phosphatase staining (ALP) and quantification (qALP) as described previously (Haimi et al., J Biomed Mater Res Part A, In press; Lindroos et al., Biochem Biophys Res Commun 2008; 368:329-35). Briefly, in qALP analysis, cells were collected in 0.1% Triton buffer (Sigma) and were frozen to −70° C. to lyse the cells. The subsequent steps of the qALP measurement were performed according to the Sigma 104 Procedure (#104-LL). For ALP purposes, the cell cultures were fixed with a 4% paraformaldehyde solution (PFA) and stained with the leukocyte alkaline phosphatase kit according to the Sigma Procedure No. 86 (#86R-1KT).

Osteogenic differentiation was evident in both osteogenically induced and control cell cultures in patient 1. Compared to the control culture, more prominent ALP staining could be detected in the osteogenically induced cultures. The qALP analysis exhibited corresponding development, where the alkaline phosphatase activity was 2.6 times greater in the differentiation cultures, compared to the control culture.

Osteogenic differentiation capacity of ASCs from patient 1 was further studied by RT-PCR for bone-associated markers with the exclusion of beta-TCP. For this purpose, ASCs were seeded into T25 culture flasks at a density of 1×10⁴ cells/cm² in OM. Total RNA was extracted from the osteogenic differentiation cell cultures with Eurozol (Euroclone S.p.A, Pero, Italy). First-strand cDNA syntheses were performed by High Capacity cDNA Archive Kit (Applied Biosystems, Warrington, UK). Real-time quantitative PCR (qPCR) was conducted using the primers Runx2 (early osteogenic marker), collagen type I (COLL-1), (extracellular matrix protein), osteocalcin (OC), osteopontin (OP), and RPLPO (human acidic ribosomal phosphoprotein P0) Primer sequences have been described previously (Lindroos et al., ibid.)(Table 2). The PCR mixture contained 50 ng cDNA, 250 nM PCR primer, and Power SYBR Green PCR Master Mix (Applied Biosystems). The reactions were performed with AbiPrism 7000 Sequence Detection System (Applied Biosystems) at 95° C. 10 min, and then 45 cycles at 95° C./15 s and 60° C./60 s. The mRNA values for Runx2, COLL-1, OC, and OP were normalized to that of the housekeeping gene RPLPO. The expression levels of the differentiation cultures were further compared with the expression levels of the control cultures. qPCR results confirmed elevated expression levels of bone associated markers RUNX2 and OC, while OP and COLL-1 showed lower expression levels in the differentiation cultures compared to the control cultures.

TABLE 2
Primers for real-time quantitative PCR

| | | | Product | Accession |
|---|---|---|---|---|
| Name | | 5′-Sequence-3′ | size | Number |
| RUNX2 | Forward | CCCGTGGCCTTCAAGGT | | |
| | | (SEQ ID NO: 1) | 76 | NM_004348 |
| | Reverse | CGTTACCCGCCATGACAGTA | | |
| | | (SEQ ID NO: 2) | | |
| Collagen type 1 | Forward | CCAGAAGAACTGGTACATCAGCAA | | |
| | | (SEQ ID NO: 3) | 94 | NM_000088 |
| | Reverse | CGCCATACTCGAACTGGAATC | | |
| | | (SEQ ID NO: 4) | | |
| Osteocalcin | Forward | AGCAAAGGTGCAGCCTTTGT | | |
| | | (SEQ ID NO: 5) | 63 | NM_000711 |
| | Reverse | GCGCCTGGGTCTCTTCACT | | |
| | | (SEQ ID NO: 6) | | |
| Osteopontin | Forward | GCCGACCAAGGAAAACTCACT | | |
| | | (SEQ ID NO: 7) | 71 | J04765 |
| | Reverse | GGCACAGGTGATGCCTAGGA | | |
| | | (SEQ ID NO: 8) | | |
| RPLPO | Forward | AATCTCCAGGGGCACCATT | | |
| | | (SEQ ID NO: 9) | 70 | NM_001002 |
| | Reverse | CGTTGGCTCCCACTTTGT | | |
| | | (SEQ ID NO: 10) | | |

In addition to the osteogenic differentiation capacity, ASCs from patient 1 were able to differentiate towards adipogenic and chondrogenic cell lineages. For the adipogenic differentiation, ASCs were seeded onto a 12-well culture plate at a density of 1×10⁴ cells/cm² in BM supplemented 33 μM biotin (Sigma), 1 μM dexamethasone, 100 nM insulin (Invitrogen) and 17 μM pantothenate (Fluka, Buchs, Switzerland). Upon seeding of cells, 250 μM iso-butylmethylxanthine (IBMX; Sigma) was added to the differentiation cultures and was removed from the culture after 24 h. The differentiation cultures were fixed with a 4% PFA prior to a staining procedure with 0.3% Oil Red O-solution (Sigma). Adipogenic differentiation was evident in both induced and control cell cultures, especially when cells were cultured in high densities. Lipid droplets started to accumulate already three days after adipogenic induction initiation and droplets enlarged steadily until the end of cell culture period.

The chondrogenic differentiation capacity was assessed by a micromass culture method as previously described (Denker et al., Differentiation 1995; 59:25-34; Zuk et al., Tissue Eng 2001; 7:211-28). Briefly, 1×10⁵ cells were seeded onto a 24-well culture plate in a 10 μl volume, and were let adhere for 3 h in a cell incubator prior to addition of the chondrogenic culture media (BM supplemented with ITS₊1 (Sigma), 50 μM asc.ac, 55 μM sodium pyruvate (Invitrogen), L-proline 23 μM (Sigma), L-glutamine, and 0.3% a/a). 10 ng/ml TGF-β (Sigma) was added to the differentiation cultures upon first change of culture media. The differentiation cultures were fixed with a 4% PFA prior to a staining procedure with 1% Alcian blue (Sigma). Chondrogenic differentiation, as shown by formation of nodule structures and Alcian blue staining, was seen in chondrogenic differentiation cultures but not in control cell cultures.

### Example 2

### Maxillary Reconstruction of Patient 1

The patient was 65-year-old male, who underwent a hemimaxillectomy 28 months earlier due to a large recurrent keratocyst. The orbital floor had been reconstructed with a calvarial bone graft covered with temporal muscle. During the follow-up, the patient had a removable obturator prosthesis in the area and no signs of recurrence were noted.

In the first operation approximately 200 ml of subcutaneous abdominal fat was harvested, and 60 ml of autologous serum was collected. The fat and serum were transported to REGEA for stem cell isolation and expansion as described in Example 1.

The second operation was performed in general anesthesia 16 days after the first operation. In this operation, a preformed titanium cage filled with ASCs and beta-TCP was inserted carefully through vertical incision in a pouch prepared in the patient's left rectus abdominis muscle. Care was taken not to disturb the vascular supply of the muscle. The incision was closed in layers. Prophylactic cefuroxime (Zinacef®, GlaxoSmithKline, Espoo Finland) 1.5 g was administered three times a day for two days intravenously (i.v.) and subsequently cephalexin (Kefexin® Orion, Espoo, Finland) 500 mg three times a day orally (p.o.) for one week. The patient was discharged from the hospital on the second day p.o. and was followed up clinically and radiologically for 8 months before the third operation.

In the third operation, the rectus abdominis free flap was raised. Prior to disconnecting the vascular supply to the muscle, the muscle pouch was carefully opened and the titanium cage was opened. The bone neotissue was macroscopically examined and a small biopsy of the bone was taken. After disconnecting the vessels, the flap was placed in the maxillary defect; inferior epigastric artery was anastomosed end-to-end to facial artery and vein end-to-end to facial vein. The muscle was left to epithelialize intraorally. Due to an unexpected occlusion of the vein, the patient was reoperated on the first day p.o. and the flap was salvaged. Prophylactic cefuroxime (Zinacef®) 1.5 g and metronidazole (Metronidazole Braun®, Melsungen, Germany) 500 mg three times a day i.v. for one week was initiated and thereafter one week of cephalexin (Kefexin®) 500 mg and metronidazole (Trikozol®, Orion) 400 mg three times a day p.o. were administered. Patient was discharged on the 10th day p.o.

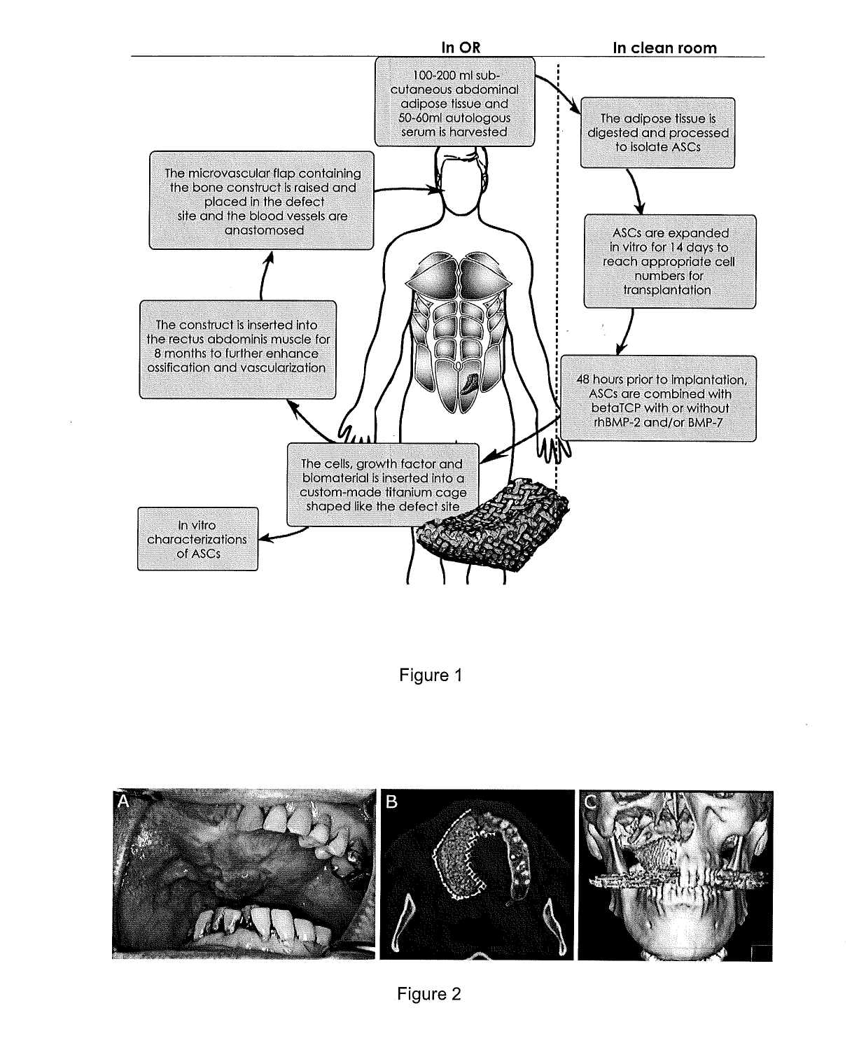

The patient was followed up clinically between the second and third operation for 8 months. Clinically, the patient's healing was uneventful. Radiological follow-up included skeletal scintigraphy and abdominal CT. When the flap was raised and bone biopsy was taken, the bone neotissue was vital and vasculature had developed within the implant. The bone resembled mature bone. Two months after the reconstruction operation, the rectus abdominis muscle had atrophied almost completely and the surface had epithelialized apart from a small area in the molar region (FIG. 2 A). Radiologically, the bone resembled mature maxillary bone (FIGS. 2 B and 2C). After the third operation the patient has been followed up clinically and radiologically for 12 months.

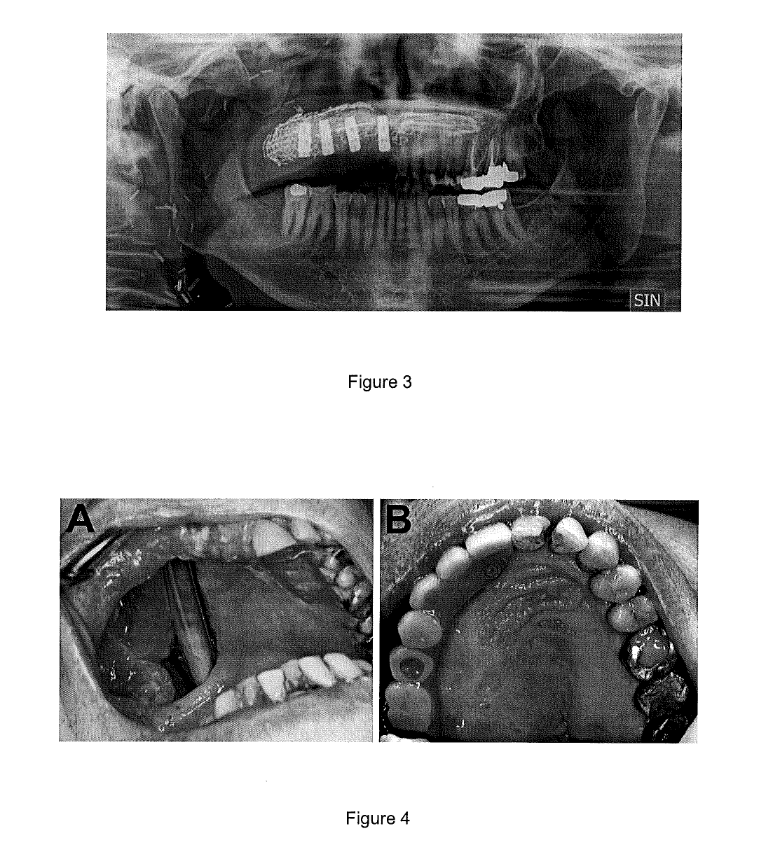

Four dental implants (OsseoSpeed™ 5.0, Astra Tech, Mölndal, Sweden) were placed four months after the third operation, with excellent primary stability (FIG. 3). The implants osseointegrated without any adverse events and the dental rehabilitation was carried out with a fixed screw retained (temporary) acrylic bridge with UniAbutments™ (Astra Tech) seven months later. Occlusion was adjusted for reduced contact in centric occlusion and articulation movements to allow gradual loading of the bone regenerate. The regeneration of the palatal mucosa was also outstanding (FIG. 4 B).

### Example 3

### Maxillary Reconstruction of Patient 2

The patient was a 59-year old male, who had undergone a complete tooth extraction of the maxilla 30 years ago. The patient was suffering from inadequately fitting dentures.

In the first operation, approximately 200 ml of subcutaneous abdominal fat was harvested and 60 ml of autologous serum was collected. The fat and serum were transported to REGEA for stem cell isolation and expansion as described in Example 1.

In the second operation, the stem cells and beta-TCP were put in the titanium cage which was shaped using the stereolithography model. The left latissimus dorsi muscle was exposed through a lateral thoracic incision. The muscle was separated laterally and its vascular pedicle (thoracodorsal artery and venae comitantes) was identified. Three to four cm distally to the area were the pedicle runs into to latissimus dorsi muscle the muscle was open laterally and the pouch for the cage with ASCs and beta-TCP was formed. The cage with dental implants was inserted so that the occlusal plane was toward the chest wall. Care was taken not to disturb the vascular supply of the muscle. The incision was closed in layers.

The patient was followed up clinically between the second and third operation for 8 months. Clinically, the patient's healing was uneventful. Radiological follow-up included skeletal scintigraphy and abdominal CT

Seven months later, in the third operation, the muscle was harvested in the normal manner and the titanium cage was opened cranially and fixed to the maxilla with 1.5 titanium screws. Vascular anastomosis were done to the facial artery and vein end-to-end. The latissimus dorsi free flap was raised and the jaw regenerate was successfully placed in the maxillary defect area, without any unexpected complications. Shortly after the surgical procedure, the jaw regenerate was lost, not due to the quality of the regenerate, but due to insufficient ossification. The jaw regenerate in Patient 2 had ossified to a lesser extent than in the case of Patient 1.

### Example 4

### Maxillary Reconstruction of Patient 3

The third maxillary reconstruction described herein is carried out for a patient with a jaw bone deficiency. The harvesting of adipose tissue is carried out as in Example 1. In the first operation, approximately 150 ml of subcutaneous abdominal fat is harvested and 55 ml of autologous serum obtained for the expansion of the autologous stem cells. All surgical operations and in vitro expansions are carried out paying due attention to sterility and GMP criteria.

ASCs are isolated, expanded and prepared for cell transplantation in vitro in GMP-class clean rooms according to Standard Operation Procedures at REGEA Institute for Regenerative Medicine as in Example 1. The beta-TCP and cells are combined like in Example 1. Cell sterility and endotoxins is tested at the Department of Public Health (University of Helsinki, Helsinki, Finland) according to methods described in European Pharmacopoeia (Council of Europe, Strasbourg) and the cells are tested negative for mycoplasma contamination as determined by a mycoplasma PCR kit (VenorGem, Minerva Biolabs GmbH, Berlin, Germany).

ASCs are expanded in vitro and analyzed by flow cytometry (FACSAria®, BDBiosciences, Erembodegem, Belgium). Monoclonal antibodies against CD9-PE, CD10-PECy7, CD13-PE, CD29-APC, CD49d-PE, CD90-APC, CD106-PE-Cy5 and CD166-PE (BD Biosciences); CD45-FITC (Miltenyi Biotech, Bergisch Gladbach, Germany); CD31-FITC, CD34-APC and CD44-FITC (Immunotools GmbH Friesoythe, Germany); and CD105-PE (R&D Systems Inc, MN, USA) are used. The surface marker analysis demonstrates that the ASC population from Patient 3 has homogenous morphology and shows a surface marker profile characteristic of ASCs with positive expression for adhesion molecules CD9, CD29, CD49d, CD105, CD106, and CD166; receptor molecule CD44; surface enzymes CD10 and CD13, and extracellular matrix protein CD90, and lack of CD31, CD34, CD45, and CD106 expressions suggesting lack of cells of hematopoietic and angiogenic lineages. Furthermore, multipotent differentiation capacity is evaluated as described in example 1.

In the second operation, the combination of stem cells and biomaterial is placed in a titanium cage which is shaped using a stereolithography model. The intramuscular ossification with dental implants is done as described in example 2.

The patient is followed up clinically between the second and third operation for 6 to 9 months. Clinically, the patient's healing is uneventful. Radiological follow-up includes skeletal scintigraphy and abdominal CT to follow-up the ossification of the regenerate. Furthermore, ossification is analyzed from histological biopsies taken in the third operation.

Six to nine months later, in the third operation, the muscle is carefully harvested and the titanium cage opened cranially and fixed to the maxilla with 1.5 titanium screws. The dental implants are provided with temporary acrylic prosthesis, and vascular anastomosis is done to the facial artery and vein end-to-end. The latissimus dorsi free flap is raised and the jaw regenerate successfully placed in the maxillary defect area, without any unexpected complications.

Later, the temporary prosthesis is replaced with a final prosthesis.

1. A biological jaw regenerate comprising a biocompatible scaffold inoculated with osteogenic cells.

2. The regenerate according to claim 1 further comprising at least one dental implant for attaching dental prosthesis.

3. The regenerate according to claim 1, wherein the scaffold comprises a frame filled with a bone substitute material.

4. The regenerate according to claim 3, wherein the frame is an inert metal cage and the bone substitute material is beta-TCP.

5. The regenerate according to claim 3, wherein the scaffold is a homogenous structure made of a biocompatible material.

6. The regenerate according to claim 1, wherein the osteogenic cells are mesenchymal stem cells.

7. The regenerate according to claim 6, wherein the mesenchymal stem cells have been obtained from autogenic fat.

8. The regenerate according to claim 1, further comprising at least one osteoinductive growth factor.

9. A method of preparing a jaw regenerate according to claim 1, comprising:
a) providing a biocompatible scaffold shaped to fit into the jaw region where the regenerate is required, and
b) inoculating said scaffold with osteogenic cells to form a biological jaw regenerate.

10. The method according to claim 9, wherein the scaffold comprises fastened thereto at least one dental implant for a dental prosthesis.

11. The method according to claim 10, further comprising adding an osteoinductive growth factor to the regenerate.

12. A method of reconstructing a jaw defect in a patient in need thereof, comprising:
a) providing a biological jaw regenerate according to claim 1,
b) inserting the biological jaw regenerate into a pouch in an autogenic muscle,
c) allowing formation of microvasculature in the biological jaw regenerate,
d) relocating the regenerate and its microvasculature to the jaw region where the regenerate is required.

13. The method according to claim 12 wherein the scaffold comprises fastened thereto at least one dental implant for a dental prosthesis, and wherein the dental prosthesis is attached to the dental implant between steps c) and d).
